(19) United States
(12) Patent Application Publication
(10) Pub. No.: US 2021/0338236 A1
(43) Pub. Date: Nov. 4, 2021

(54) THREAD LIFT CANNULA AND METHOD OF PERFORMING A THREAD LIFT

(71) Applicant: Greene Technologies, LLC, Cave Creek, AZ (US)

(72) Inventor: David Greene, Cave Creek, AZ (US)

(21) Appl. No.: 16/862,454

(22) Filed: Apr. 29, 2020

(51) Int. Cl. A61B 17/06

(52) U.S. Cl. CPC A61B 17/06166; A61B 2017/00792

(57) ABSTRACT

A thread lift cannula has a plurality of therapeutic liquid discharge ports to enable the discharge of a therapeutic liquid into the surrounding tissue. The therapeutic liquid may be injected into the thread lift cannula conduit by a syringe and the thread therein may be bioresorbable and absorb the therapeutic liquid. The therapeutic liquid may include a medicament to reduce swelling or to treat pain, or a biologic that aids in collagen creation, formation, or restructuring. The plurality of therapeutic liquid discharge ports may vary in size along the length of the cannula. A distal discharge port may be larger than a proximal discharge port to enable a more uniform delivery of therapeutic liquid along the cannula. A therapeutic liquid discharge port may be configured at a port angle, offset from orthogonally through the wall of the cannula, to prevent blockage of the outlet opening of the discharge port.

### BACKGROUND

### Field

The disclosure relates to a thread lift cannula having a plurality of therapeutic liquid discharge ports and a method of performing a thread lift with said thread lift cannula.

### Background

Thread lifts are performed to restructure tissue, oftentimes in the face area. A thread lift places a suture, such as a bioresorbable suture, in the tissue which creates a biological reaction in the native tissue consistent with a healing cascade for wounds. This includes increased collagen and elastin formation, which offers the result of wrinkle reduction and skin tightening. This creates an aesthetic benefit for recipients.

### SUMMARY

The disclosure is directed to a thread lift cannula having a plurality of therapeutic liquid discharge ports and a method of performing a thread lift with said thread lift cannula. An exemplary thread lift cannula system includes a thread lift cannula having a therapeutic liquid inlet port for receiving a therapeutic liquid that is delivered along the cannula conduit and out into the tissue through a plurality of therapeutic liquid discharge ports. The therapeutic liquid may coat the thread that is configured in the tissue and may slowly release from the thread over time. An exemplary thread may be a resorbable thread that readily accepts the therapeutic liquid. The therapeutic liquid may include a medicament to reduce swelling or to treat pain, or a biologic that aids in collagen creation, formation, or restructuring.

An exemplary cannula of the thread lift cannula system has a proximal end and a distal end, that is inserted into the tissue. The distal end may be a blunt tip that facilitates the location of the cannula under the tissue with reduced risk of puncture or damage over a needle. The exemplary cannula has a cannula conduit extending from a location near the proximal end toward the distal end. The plurality of therapeutic liquid discharge ports are in fluid communication with the cannula conduit and extend through the cannula along a length axis to enable discharge of a therapeutic liquid into the tissue. The length axis of a therapeutic liquid discharge port may be configured at a port angle to direct the therapeutic liquid out along the cannula and this angle may facilitate discharge from the therapeutic liquid discharge port. A therapeutic liquid discharge port that is orthogonal to the cannula conduit or the outer surface of the cannula may require higher pressures to discharge the fluid. The tissue pressing on the outside of the cannula may more easily create a seal along the opening of an orthogonal therapeutic liquid discharge port, thereby making it more difficult to discharge the therapeutic liquid. An exemplary port angle may be about 10 degrees or more, about 20 degrees or more, about 40 degrees or more about 70 degrees or less and any range between and including the port angles provided, such as from about 10 degrees to about 70 degrees. A port angle may be an angle that is not perpendicular to the length of the cannula, and may create a non-orthogonal path for fluid to flow from the cannula conduit through the port and into tissue of the patient.

The plurality of therapeutic liquid discharge ports may vary in size along the cannula conduit and ports closer to the proximal end may be smaller in size than those closer to the distal end to provide a more uniform discharge of therapeutic liquid along the length of the cannula conduit. The pressure of the therapeutic liquid will drop from the proximal end to the distal end and a gradual increase in the size of the therapeutic liquid discharge ports along the cannula may provide more uniform discharge. A proximal therapeutic liquid discharge port may be smaller than a distal therapeutic liquid discharge port wherein the distal therapeutic liquid discharge port is at least 10% greater in cross-sectional area than the proximal therapeutic liquid discharge port, or at least 20% greater in cross-sectional area, or at least 40% greater in cross-sectional area, and any range between and including the percentages provided.

An exemplary thread lift cannula system locates a thread in the tissue and this thread may be configured in the cannula conduit before insertion into the tissue. The thread may extend from a proximal thread port to a distal thread port. The distal end of the thread may be retained in the tissue and as the cannula is extracted, the thread may slide through the cannula conduit and remain in the tissue.

An exemplary thread may be a bioresorbable thread including but not limited to, polydioxanone (PDO). The bioresorbable thread may include one or more types of bioresorbable materials, including, but not limited to polymers, metals, ceramics, glasses, and biological material, such as natural collagen or birth tissue. Biological material may be processed and used in the thread as a core portion of the thread or as a coating on the thread. The thread may be pre-soaked in a therapeutic liquid or may be coated with therapeutic liquid, as described herein, in situ while in the thread lift cannula. The therapeutic liquid may be dispensed into the thread lift cannula conduit and the therapeutic liquid may absorb into the thread, therein.

An exemplary thread lift cannula system may include a syringe containing a therapeutic liquid that is configured to dispense the therapeutic liquid into the cannula conduit and over the thread and out of the plurality of therapeutic ports. The syringe may be configured to dispense the therapeutic liquid into the therapeutic liquid inlet port and may have a dispensing end adapter that is configured to seat and/or seal with the therapeutic liquid inlet port.

A method of using an exemplary thread lift cannula system for a thread lift procedure includes utilizing a thread lift cannula, and thread as described herein. The thread lift cannula may be inserted in tissue to produce a tissue conduit. The thread may be configured in the cannula conduit and may extend out from the proximal thread port. The distal end of the thread may be retained in position until the thread lift cannula is extracted from the tissue. A therapeutic liquid may be dispensed, such as by injection through the therapeutic liquid inlet port to coat the thread and to dispense out of the plurality of therapeutic liquid discharge ports. As described herein, the thread may be a bioresorbable thread that may absorb the therapeutic liquid.

The summary is provided as a general introduction to some of the embodiments and is not intended to be limiting. Additional example embodiments including variations and alternative configurations are provided herein.

### BRIEF DESCRIPTION OF THE DRAWINGS

The accompanying drawings are included to provide a further understanding of the disclosure provided and are incorporated in and constitute a part of this specification, illustrate embodiments of the disclosure, and together with the description serve to explain the principles of the disclosure.

FIG. 1 shows a cross-sectional view of an exemplary thread lift cannula having a plurality of therapeutic liquid discharge ports and a thread extending along the cannula conduit.

FIG. 2 shows the profile of a person having two areas indicated for a thread lift.

FIG. 3 shows an exemplary thread lift cannula being inserted into the person's tissue.

FIG. 4 shows the exemplary thread lift cannula of FIG. 3 now inserted into the person's tissue with the proximal end of the thread extending out from the proximal thread port.

FIG. 5 shows the exemplary thread lift cannula of FIG. 4 with a syringe coupled with the therapeutic inlet port for dispensing the therapeutic liquid into the cannula conduit and over the thread and out through the therapeutic liquid discharge ports.

FIG. 6 shows an exemplary thread lift cannula configured in tissue and a syringe coupled with the therapeutic inlet port for dispensing the therapeutic liquid into the cannula conduit and over the thread and out through the therapeutic liquid discharge ports.

FIG. 7 shows the tissue upon removal of the thread lift cannula being coated with the therapeutic liquid.

Corresponding reference characters indicate corresponding parts throughout the several views of the figures. The figures represent an illustration of some of the embodiments of the present disclosure and are not to be construed as limiting the scope of the invention in any manner. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present disclosure.

### DETAILED DESCRIPTION

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the disclosure. This description should be read to include one or at least one and the singular also includes the plural unless it is obvious that it is meant otherwise.

Certain exemplary embodiments of the present disclosure are described herein and are illustrated in the accompanying figures. The embodiments described are only for purposes of illustrating the present disclosure and should not be interpreted as limiting the scope of the invention. Other embodiments of the disclosure, and certain modifications, combinations, and improvements of the described embodiments, will occur to those skilled in the art and all such alternate embodiments, combinations, modifications, improvements are within the scope of the present invention.

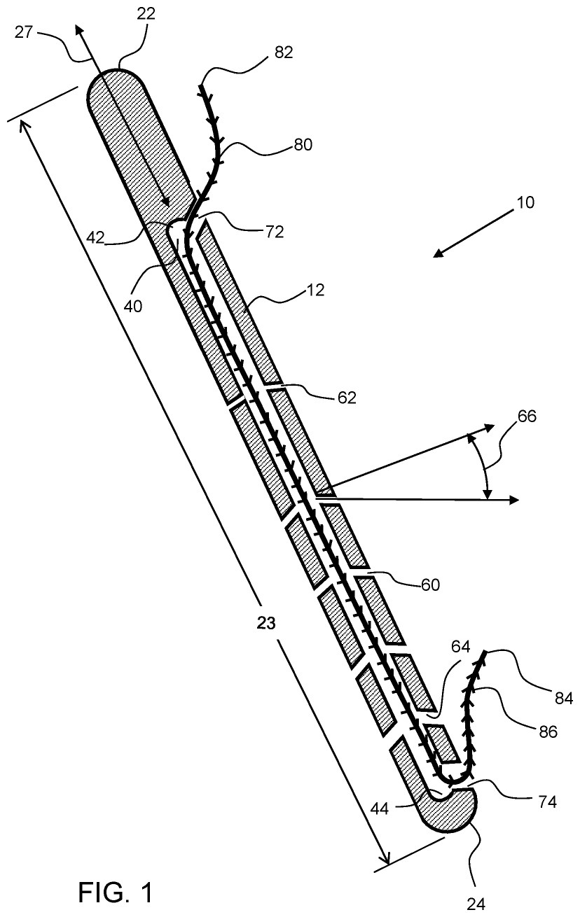

As shown in FIG. 1, an exemplary thread lift system 10 utilizes a thread lift cannula 12 that has a plurality of therapeutic liquid discharge ports 60 and a thread 80 extending along the cannula conduit 40. The thread lift cannula has a length 23 from a cannula proximal end 22 to a cannula distal end 24 and a length axis 27 extending along the length of the thread lift cannula. The cannula conduit 40 extends a length from a conduit proximal end 42 to a conduit distal end 44. The thread extends through the cannula conduit 40 and has a thread proximal end 82 extending out from the proximal thread port 72 and a thread distal end 84 that extends out from distal thread port 74. A thread may be a bioresorbable thread that has a plurality of barbs 86 that extend out from the thread to retain the thread in the tissue and prevent migration or movement of the thread upon implant. A plurality of therapeutic liquid discharge ports 60 are configured in the cannula 12 and extend out from the cannula conduit 40. The therapeutic liquid discharge ports 60 may increase in size from a proximal therapeutic liquid discharge port 62 to a distal therapeutic liquid discharge port 64 to provide more uniform dispensing of therapeutic liquid therefrom. The pressure of the therapeutic liquid will decrease down along the cannula conduit 40 from the conduit proximal end 42 to the conduit distal end 44 and therefore a larger therapeutic liquid discharge ports on the conduit distal end may provide a similar discharge of the therapeutic liquid to that of a smaller and more proximal therapeutic liquid discharge port. Some or all of the therapeutic liquid discharge ports may be configured at a port angle 66 wherein the port extends, through the wall of the thread lift cannula at a port angle from orthogonal to the length axis of the thread lift cannula.

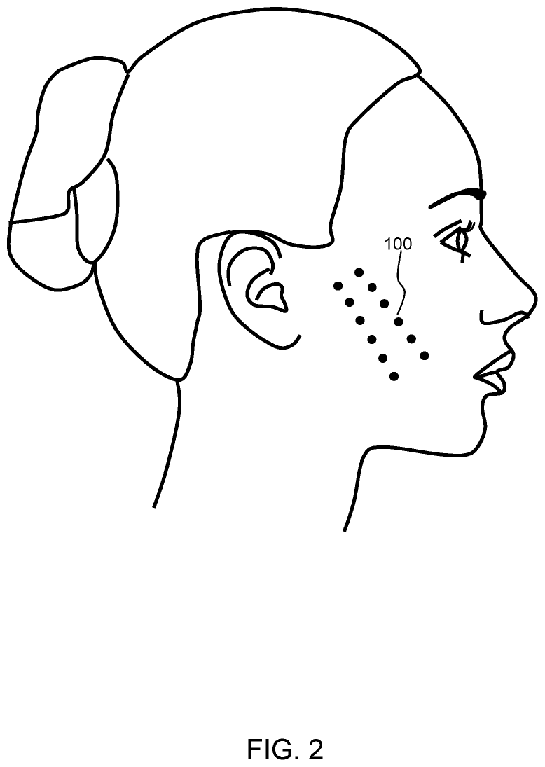

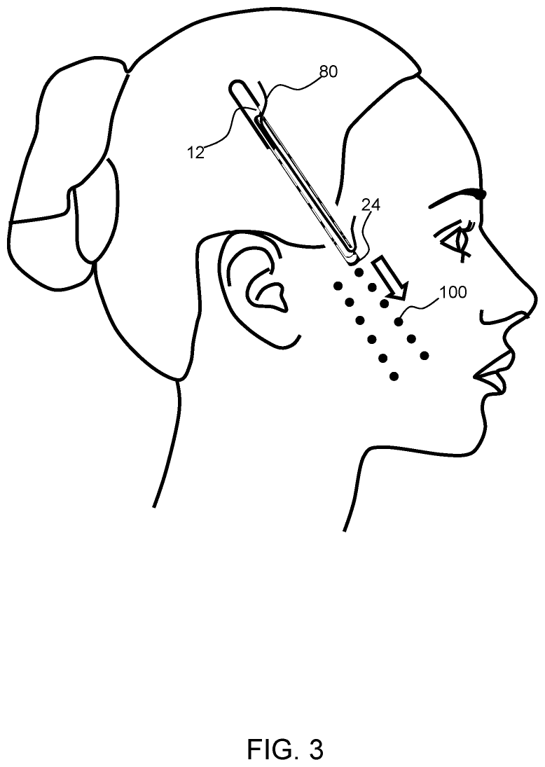

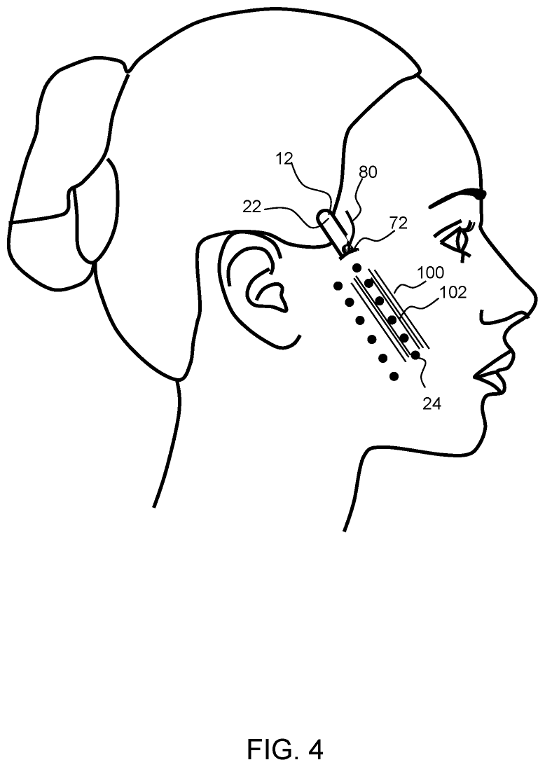

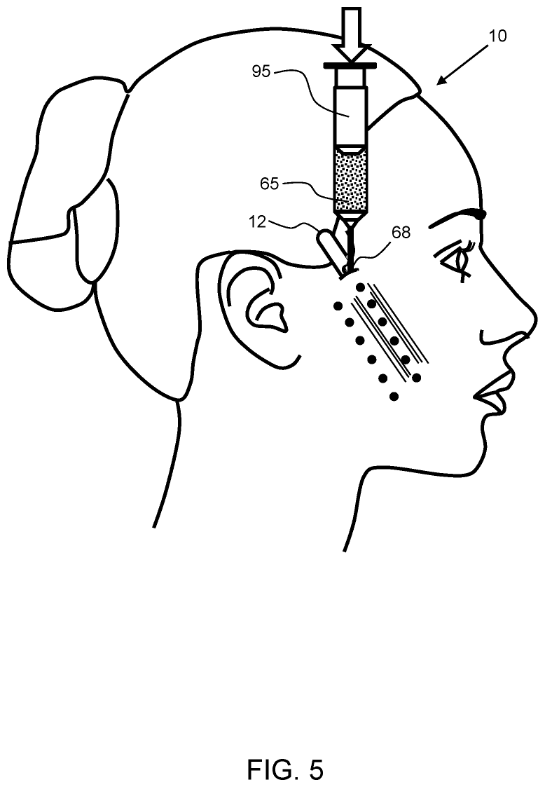

Referring now to FIGS. 2 to 5, a person has a thread lift preformed along their face tissue 100. As shown in FIG. 2, two areas are indicated for a thread lift along their face tissue 100 as indicated by the markings on their face. As shown in FIG. 3, the cannula distal end 24 is inserted into the tissue to form a tissue conduit 102, as depicted in FIG. 4. As shown in FIG. 4, the cannula proximal end 22 is exposed and the thread 80 extends out from the proximal thread port 72. As shown in FIG. 5, a syringe 95 is coupled with the therapeutic liquid inlet port 68 to dispense the therapeutic liquid 65 into the cannula conduit 40 of the thread lift cannula 12. The thread 80 is therefore coated with the therapeutic liquid in the therapeutic thread lift system 10.

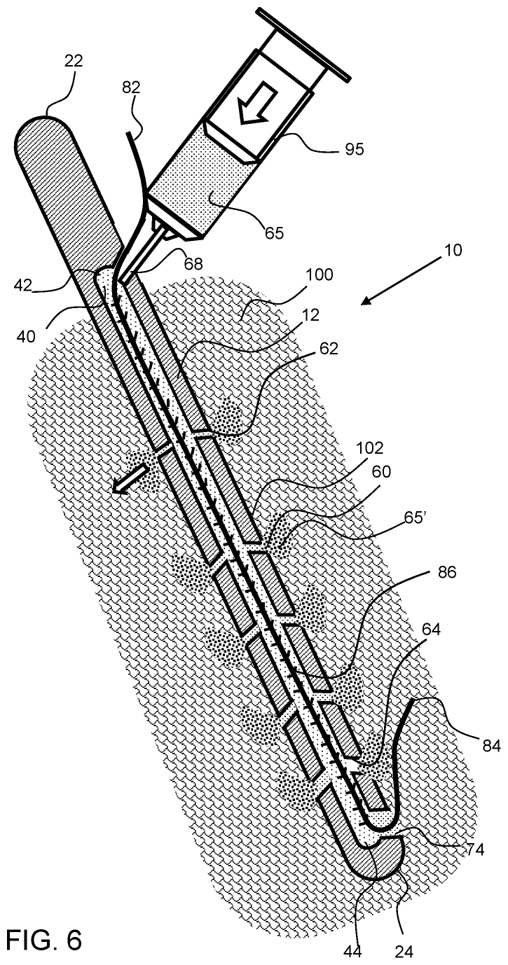

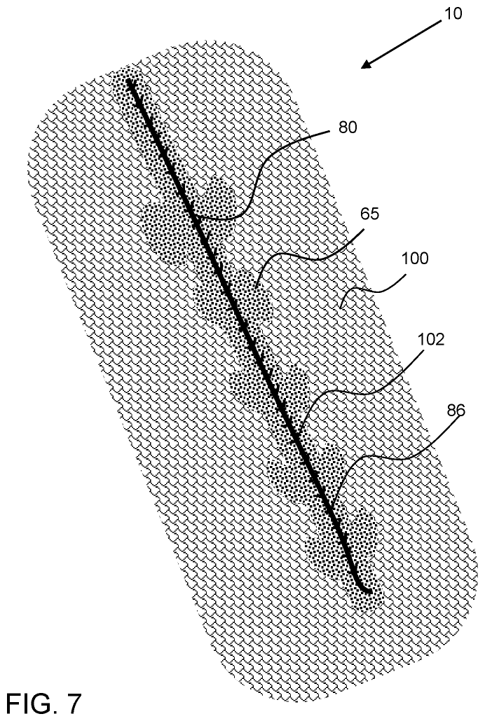

Referring now to FIGS. 6 and 7, an exemplary thread lift cannula 12 is inserted into tissue 100 to form a tissue conduit 102 in the tissue. The thread 80 extends along the cannula conduit 40 and therapeutic liquid 65 is coated onto the thread. In addition, the syringe 95, coupled with the therapeutic liquid inlet port 68, forces the therapeutic liquid 65 into the cannula conduit 40 of the thread lift cannula 12 and some of the therapeutic liquid 65′ out of the plurality of therapeutic liquid discharge ports 60. As shown in FIG. 7, the thread lift cannula 12 is removed from the tissue 100 and the tissue conduit 102 has the thread 80 and therapeutic liquid 65 therein. The barbs 86 may help to retain the thread 80 in a fixed position within the tissue and the thread may then create a biological reaction in the native tissue consistent with a healing cascade for wounds wherein there is increased collagen and elastin formation.

It will be apparent to those skilled in the art that various modifications, combinations, and variations can be made in the present invention without departing from the scope of the invention. Specific embodiments, features, and elements described herein may be modified, and/or combined in any suitable manner. Thus, it is intended that the present invention cover the modifications, combinations, and variations of this invention provided they come within the scope of the appended claims and their equivalents.

What is claimed is:

1. A thread lift cannula system comprising:
a) a cannula extending from a cannula proximal end to a cannula distal end;
b) a cannula conduit extending from a conduit proximal end to a conduit distal end;
c) a proximal thread port;
d) a distal thread port;
e) a therapeutic liquid inlet port configured proximal to the conduit proximal end;
f) a plurality of therapeutic liquid discharge ports configured along the cannula conduit;
g) a thread extending from the proximal thread port to the distal thread port; and
wherein a therapeutic liquid is configured to fill the cannula conduit and configured to dispense out of the cannula conduit through the plurality of therapeutic liquid discharge ports.

2. The thread lift cannula system of claim 1, wherein a proximal therapeutic liquid discharge port is smaller than a distal therapeutic liquid discharge port.

3. The thread lift cannula system of claim 2, wherein the distal therapeutic liquid discharge port is at least 20% greater in cross-sectional area than the proximal therapeutic liquid discharge port.

4. The thread lift cannula system of claim 1, wherein at least a portion of the plurality of therapeutic liquid discharge ports have a length axis that is configured at a port angle.

5. The thread lift cannula system of claim 4, wherein the port angle is at least 10 degrees.

6. The thread lift cannula system of claim 4, wherein the port angle is a non-orthogonal angle.

7. The thread lift cannula system of claim 1, wherein each of the plurality of therapeutic liquid discharge ports have a length axis that is configured at a port angle through the cannula.

8. The thread lift cannula system of claim 7, further comprising a syringe containing said therapeutic liquid and wherein the syringe is configured to dispense the therapeutic liquid into the therapeutic liquid inlet port to coat said thread in the cannula conduit.

9. The thread lift cannula system of claim 8, wherein the syringe is configured to force the therapeutic liquid through the plurality of therapeutic liquid discharge ports.

10. The thread lift cannula system of claim 1, wherein the thread is a bioresorbable thread.

11. A method of coating a thread during a thread lift procedure comprising:
a) providing a thread lift cannula system comprising:
i) a cannula extending from a cannula proximal end to a cannula distal end;
ii) a cannula conduit extending from a conduit proximal end to a conduit distal end;
iii) a proximal thread port;
iv) a distal thread port;
v) a therapeutic liquid inlet port configured proximal to the conduit proximal end;
vi) a plurality of therapeutic liquid discharge ports configured along the cannula conduit;
vii) the thread extending from the proximal thread port to the distal thread port;
b) placing the thread in the cannula conduit from the conduit proximal end to the conduit distal end;
c) inserting the cannula distal end into tissue;
d) providing a therapeutic liquid;
e) configuring said therapeutic liquid in the cannula conduit to produce a coated thread; and
f) removing the cannula while retaining the coated thread in the tissue.

12. The method of claim 11, further comprising providing a syringe containing said therapeutic liquid and wherein said syringe dispenses the therapeutic liquid into the cannula conduit through the therapeutic liquid inlet port.

13. The method of claim 12, wherein the syringe is coupled with the therapeutic liquid inlet port after the inserting the cannula distal end into the tissue.

14. The method of claim 13, wherein the syringe forces the therapeutic liquid from the cannula conduit out through the plurality of therapeutic liquid discharge ports.

15. The method of claim 11, wherein a proximal therapeutic liquid discharge port is smaller than a distal therapeutic liquid discharge port.

16. The method of claim 15, wherein the distal therapeutic liquid discharge port is at least 20% greater in cross sectional area than the proximal therapeutic liquid discharge port.

17. The method of claim 11, wherein at least a portion of the plurality of therapeutic liquid discharge ports have a length axis that is configured at a port angle.

18. The method of claim 11, wherein each of the plurality of therapeutic liquid discharge ports have a length axis that is configured at a port angle through the cannula.

19. The method of claim 18, wherein the port angle is at least 10 degrees.

20. The method of claim 11, wherein the thread is a bioresorbable thread.
